(19) United States
(12) Patent Application Publication
(10) Pub. No.: US 2025/0366472 A1
(43) Pub. Date: Dec. 4, 2025

(54) 1-AMINOCYCLOPROPANE-1-CARBOXYLIC ACID OXIDASE INHIBITORS (ACO-I)

(71) Applicant: Royal Holloway and Bedford New College, Surrey (GB)

(72) Inventors: George Heslop-Harrison, Surrey (GB); Robin Williams, Surrey (GB)

(73) Assignee: Royal Holloway and Bedford New College, Surrey (GB)

(21) Appl. No.: 18/877,932

(22) PCT Filed: Jul. 4, 2023
(86) PCT No.: PCT/GB2023/051754

§ 371 Date: Dec. 20, 2024

(30) Foreign Application Priority Data
Jul. 6, 2022 (GB) 2209916.2

(51) Int. Cl. A01N 35/06; A01N 3/02; A01P 21/00; C07C 49/657; C07C 69/757; C07D 239/545; C07D 307/46; C07D 307/54; C07D 307/80; C07D 309/36

(52) U.S. Cl. CPC A01N 35/06; A01N 3/02; A01P 21/00; C07C 49/657; C07C 69/757; C07D 239/545; C07D 307/46; C07D 307/54; C07D 307/80; C07D 309/36; C07C 2601/08; C07C 2601/14

(57) ABSTRACT

This invention provides for use of a compound of formula (1) for inhibiting a post-germination ethylene production response in a plant or plant part and a method of inhibiting a post-germination ethylene production response of a plant or plant part comprising delivering a compound of formula (1) to a plant or plant part.

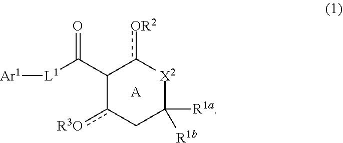

### TECHNICAL FIELD OF THE INVENTION

The present invention relates to the use of compounds for inhibiting a post-germination ethylene production response in a plant or plant part.

### BACKGROUND TO THE INVENTION

Ethylene is an important phytohormone that promotes the ripening of fruits and senescence of flowers, thereby reducing their shelf lives, but has a range of other roles relating to seed germination, plant resistance to stress, and crop sciences.

The ethylene biosynthesis pathway in plants is well understood. In short, ethylene is synthesized from S-adenosylmethionine (SAM), which is converted to 1-amino cyclopropane-1-carboxylate (ACC) by the enzyme ACC synthase (ACS). ACC is then oxidized by the ACC oxidase (ACCO or ACO, referred to as ACO herein), giving rise to ethylene, carbon dioxide and cyanide. Usually, plant ethylene production is maintained at a low basal level but is induced rapidly and dramatically under certain developmental stages or stress.

ACO acts as a control point under specific developmental and stress conditions in various plant species. The ACO enzyme is a 2OG-oxygenase ‘related’ enzyme that belongs to the cupin superfamily, which uses a non-heme ferrous iron as a cofactor and facilitates the integration of molecular oxygen into a myriad of biomolecules.

Ethylene acts at trace levels throughout the life of the plant by stimulating or regulating the ripening of fruit, the opening of flowers, and the abscission (or shedding) of leaves. Specific ethylene biosynthesis inhibitors have been suggested to help to decrease postharvest loss under normal and high stress situations such as when the plant is exposed to high heat, drought or cold temperatures. Typically known ethylene biosynthesis inhibitors require high concentrations to be affective.

There are different ways to inhibit ethylene, including inhibiting ethylene synthesis and inhibiting ethylene perception (receptors). Inhibitors of ethylene perception include compounds that have a similar shape to ethylene, but do not elicit the ethylene response.

Analogues of ACC, such as a-aminoisobutyric acid (AIB) and 2-aminooxyisobutyric acid (AOIB), inhibit ethylene formation by competitively targeting ACO, but with a low inhibition potency (Satoh and Esashi, et al. 1982; Kosugi, et al. 2014).

More recently, pyrazinamide (PZA) has been identified as an ethylene biosynthesis inhibitor. The primary action of PZA is to function as a pro-drug, and is converted to pyrazinecarboxylic acid (POA) by the mycobacterial enzyme pyrazinamidase (PZase)/nicotinamidase. PZA conversion in plants produces POA which directly binds to the ACO proteins and inhibits their enzyme activity (Sun, et al. 2017).

Despite the availability of specific ACO inhibitors, there exists a problem in that low inhibition potency/efficacy of these known inhibitors means a high concentration of inhibitor are needed to prevent ethylene production at a commercially relevant level. It would therefore be advantageous to identify highly potent compounds which inhibit an ethylene production response and therefore may be used at lower concentrations.

The global cut flower industry is a high profit, multi-billion-dollar industry. Flowers are typically cut and then transported, often long distances, before reaching their final destination wherein the desire is for the cut flowers to last as many days as possible. It would therefore be advantageous to identify highly potent compounds which prevent or slow flower senescence and may be used at lower concentrations.

Global warming is leading to most land mass experiencing high stress conditions which may impact plants, in particular, crops. It would therefore be advantageous to identify highly potent compounds which mitigate a plant's stress response and may be used at lower concentrations.

It is an aim of embodiments of the invention to overcome one or more problems of the prior art, whether expressly disclosed herein or not.

### SUMMARY OF THE INVENTION

According to a first aspect of the invention there is provided a use of a compound of formula (1) for inhibiting a post-germination ethylene production response in a plant or plant part wherein formula (1) is

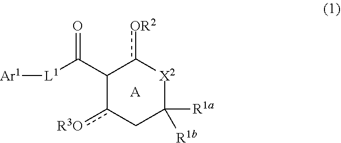

or a salt or tautomer thereof, wherein:
ring A is a six-membered aromatic or non-aromatic ring in which X¹ and X₂ are independently selected from O, CH, CH₂, CH(C₁₋₄ alkyl), and C(C₁₋₄ alkyl)₂;
R^{1a} and R^{1b} are independently selected from hydrogen and C₁₋₄ alkyl or R^{1a} and R^{1b} together form a carbonyl group with the carbon atom of ring A to which they are attached;
R² and R³ are independently selected from hydrogen and C₁₋₄ alkyl or are absent when the oxygen atom to which they are attached forms a carbonyl group with the carbon ring member of ring A;
L¹ is selected from a bond, —CH₂, —CH═CH— and —CH₂—CH₂—
Ar¹ is a 5- or 6-membered carbocyclic or heterocyclic aromatic group optionally substituted by one or more substituents R⁴; and
R⁴ is selected from hydroxy, halogen, O—Ar², Hyd¹, O—Hyd¹, NH(Hyd¹) and N(Hyd¹)₂, wherein Hyd¹ is a C₁₋₄ hydrocarbon group optionally substituted with one or more fluorine atoms; and
Ar² is a 5- or 6-membered carbocyclic or heterocyclic aromatic group.

Compounds of formula (1) may be advantageous for inhibiting a post-germination ethylene production response because they may behave as ACO inhibitors. Compounds of formula (1) may be advantageous for inhibiting a post-germination ethylene production response because they are highly potent and therefore can be used at low concentrations.

The post-germination ethylene production response may be selected from the group consisting of: preventing or slowing food ripening or crop maturation; preventing or slowing plant or plant part senescence; preventing or slowing flower senescence; improving crop quality whilst on the plant or following harvest; reducing a biotic or an abiotic stress response in a plant, for example a response to heat and drought stress; and maintaining the freshness of plants or plant parts or any combination thereof.

Reducing a biotic or an abiotic stress response in a plant may comprise improving recovery from an induced stress response. The stress response may be caused or induced by extended periods of extreme temperatures (hot or cold), frost, pollution, wind, drought, flood, salt-stress, metal-stress, nutrient-stress, ozone levels, fungal infection, bacterial infection, exposure to bacterial pathogens or other stresses or any combination of stresses.

The compounds of formula (1) may be used to improve plant health, yield, vigour, or yield quality or volume, or the levels of defined natural products in plants, or other characteristics. The compounds of formula (1) may therefore be advantageous for inhibiting a post-germination ethylene production response in a plant or plant part because they may increase the quality and/or yield of a plant crop, through blocking ethylene production which is the first step in a stress response; thereby reducing or blocking the stress response and avoiding adverse effects on plant growth, development and productivity and plant products yield, quality, and other characteristics.

The compound of formula (1) may be used in a composition comprising the compound and at least one carrier. In some embodiments the amount of the compound of formula (1) in the composition is between 0.005 μM and 50 μM. In some embodiments the concentration of the compound of formula (1) is between 0.01 μM and 50 μM, 0.05 μM and 50 μM, 1 μM and 50 μM, 0.005 μM and 40 μM, 0.01 0.005 μM and 40 μM, 0.005 μM and 30 μM, 0.01 μM and 30 μM, 0.005 μM and 20 μM, 0.01 μM and 20 μM, 1 μM and 20 μM, 5 μM and 20 μM, 0.005 μM and 15 μM, 0.01 μM and 15 μM, 0.05 μM and 15 μM, 0.1 μM and 15 μM, 1 μM and 15 μM, 5 μM and 15 μM or between 8 μM and 12 μM. This embodiment may be advantageous because the compound of formula (1) is highly potent and therefore can be used at significantly lower concentrations than known ethylene production inhibitors.

In some embodiments, the plant is selected from the group consisting of: potted plants, agricultural crops, flowers, bedding plants, nursery plants, fruits, vegetables, ornamental plants, aromatic plants, plantation plants, flowering plants and medicinal crops.

In embodiments wherein the plant is an agricultural crop, the agricultural crop may be selected from the group consisting of: rice crops, rye crops, barley crops and wheat crops or any combination thereof. The plant may be a flowering plant. The plant may be selected from the group consisting of: a carnation, a daffodil, a rose, a tulip, a lily, a chrysanthemum, an iris, a hyacinth, a dahlia, or any combination thereof. The use of compounds of formula (1) with agricultural crops may be advantageous because the compounds of formula (1) may reduce ethylene production and therefore increase starch synthesis and rice quality. The use of compounds of formula (1) with a flowering crop may be advantageous because the compounds of formula (1) may reduce flower senescence and therefore increase the lifetime of cut flowers.

In some embodiments X¹ and X² are independently selected from O, CH, CH₂, CH(CH₃), and C(CH₃)₂. In preferred embodiments X¹ and X² are independently selected from O, CH, and CH₂.

In some embodiments R^{1a} is hydrogen and R^{1b} is methyl. In some embodiments R^{1a} and R^{1b} are methyl.

In some embodiments R² and R³ are independently selected from hydrogen and methyl. In some embodiments both R² and R³ are absent such that in each case the oxygen atom forms a carbonyl group with the carbon ring member of ring A.

In some embodiments Ar¹ is a 6-membered carbocyclic aromatic group optionally substituted by one or more substituents R⁴. In some embodiments Ar¹ is a 6-membered heterocyclic aromatic group optionally substituted by one or more substituents R⁴.

In some embodiments the compound is 2,2,4-trimethyl-6-(3 phenylpropanoyl) cyclohexane-1,3,5-trione. 2,2,4-trimethyl-6-(3-phenylpropanoyl) cyclohexane-1,3,5-trione is also known as Myrigalone A (MyA). In some embodiments the compound is not 2,2,4-trimethyl-6-(3 phenylpropanoyl) cyclohexane-1,3,5-trione. 2,2,4-trimethyl-6-(3-phenylpropanoyl) cyclohexane-1,3,5-trione is also known as Myrigalone A (MyA).

In some embodiments the compound is selected from the group consisting of:
(a) 5-benzoyl-4,5,6,7-tetrahydro-1-benzofuran-4-one;
(b) methyl 5-acetyl-2,2-dimethyl-4,6-dioxocyclohexane-1-carboxylate;
(c) 6-amino-5-{2-[(3,4-dichlorophenyl)amino]acetyl}-1,3-dimethyl-1,2,3,4-tetrahydropyrimidine-2,4-dione;
(d) 2,6-dimethoxyphenyl 3-(5-methylfuran-2-yl)prop-2-enoate;
(e) 3-[3-(3-bromo-4-fluorophenyl)prop-2-enoyl]-6-methyl-3,4-dihydro-2H-pyran-2,4-dione;
(f) 6-methyl-3-[3-(2-methylphenyl)prop-2-enoyl]-3,4-dihydro-2H-pyran-2,4-dione;
(g) 5,5-dimethyl-2-(2-phenylacetyl)cyclohexane-1,3-dione;
(h) 2-benzoyl-5,5-dimethylcyclohexane-1,3-dione;
(i) 2-acetyl-5-phenylcyclohexane-1,3-dione;
(j) 3-[3-(4-tert-butylphenyl)prop-2-enoyl]-6-methyl-3,4-dihydro-2H-pyran-2,4-dione;
(k) 2-[(2E)-3-(furan-2-yl)prop-2-enoyl]-2,3-dihydro-1H-indene-1,3-dione;
(l) 3-[(2E)-3-[2-(difluoromethoxy)phenyl]prop-2-enoyl]-4-hydroxy-6-methyl-2H-pyran-2-one;
(m) 3-[3-(4-ethylphenyl)prop-2-enoyl]-6-methyl-3,4-dihydro-2H-pyran-2,4-dione;
(n) 3-[3-(2,3-dichlorophenyl)prop-2-enoyl]-6-methyl-3,4-dihydro-2H-pyran-2,4-dione;
(o) 3-[(2E)-3-(4-fluorophenyl)prop-2-enoyl]-4-hydroxy-6-methyl-2H-pyran-2-one;
(p) 3-{3-[4-(dimethylamino)phenyl]prop-2-enoyl}-6-methyl-3,4-dihydro-2H-pyran-2,4-dione;
(q) 3-[3-(2,5-dimethylphenyl)prop-2-enoyl]-6-methyl-3,4-dihydro-2H-pyran-2,4-dione;
(r) 2-(3-oxo-3-phenylpropyl)cyclopentan-1-one;
(s) 3-{3-[3-(4-chlorophenoxy)phenyl]prop-2-enoyl}-6-methyl-3,4-dihydro-2H-pyran-2,4-dione;
(t) 3-[3-(3-fluorophenyl)prop-2-enoyl]-6-methyl-3,4-dihydro-2H-pyran-2,4-dione; and
(u) 3-[(2E)-3-[4-(difluoromethoxy)phenyl]prop-2-enoyl]-4-hydroxy-6-methyl-2H-pyran-2-one.

Compounds (a) to (u) are of the formula shown in table 1.

TABLE 1

| Compound | RHUL | Compound | Compound |
|---|---|---|---|
| Identifier | Reference | IUPAC | Structure |
| (a) | ACOi-77-7 | 5-benzoyl-4,5,6,7- tetrahydro-1-benzofuran- 4-one | 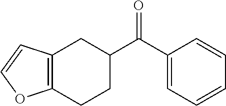 [SMILES: O=C(c1ccccc1)C1CCc2occc2C1] |
| (b) | ACOi-67-8 | methyl 5-acetyl-2,2- dimethyl-4,6- dioxocyclohexane-1- carboxylate | 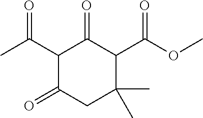 [SMILES: COC(=O)C1C(=O)C(C(C)=O)C(=O)CC1(C)C] |
| (c) | ACOi-69-10 | 6-amino-5-{2-[(3,4- dichlorophenyl)amino] acetyl}-1,3-dimethyl- 1,2,3,4- tetrahydropyrimidine- 2,4-dione | 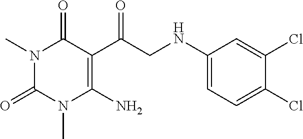 [SMILES: Cn1c(N)c(C(=O)CNc2ccc(Cl)c(Cl)c2)c(=O)n(C)c1=O] |
| (d) | ACOi-54-11 | 2,6-dimethoxyphenyl 3- (5-methylfuran-2- yl)prop-2-enoate | 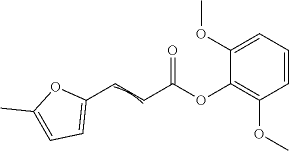 |
| (e) | ACOi-74-12 | 3-[3-(3-bromo-4- fluorophenyl)prop-2- enoyl]-6-methyl-3,4- dihydro-2H-pyran-2,4- dione | 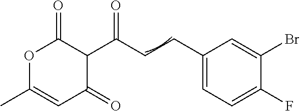 [SMILES: CC1=CC(=O)C(C(=O)CCc2ccc(F)c(Br)c2)C(=O)O1] |
| (f) | ACOi-74-12-2 | 6-methyl-3-[3-(2- methylphenyl)prop-2- enoyl]-3,4-dihydro-2H- pyran-2,4-dione | 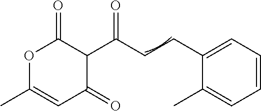 [SMILES: CC1=CC(=O)C(C(=O)CCc2ccccc2C)C(=O)O1] |
| (g) | ACOi-84-16-4 | 5,5-dimethyl-2-(2- phenylacetyl)cyclohexane- 1,3-dione | 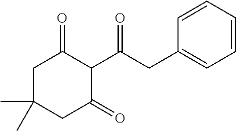 [SMILES: CC1(C)CC(=O)C(C(=O)Cc2ccccc2)C(=O)C1] |
| (h) | ACOi-0-0-7 and ACOi-68-6-3 | 2-benzoyl-5,5- dimethylcyclohexane- 1,3-dione | 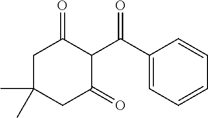 [SMILES: CC1(C)CC(=O)C(C(=O)c2ccccc2)C(=O)C1] |
| (i) | ACOi-0-0-8 | 2-acetyl-5- phenylcyclohexane-1,3- dione | 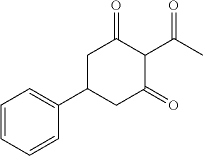 [SMILES: CC(=O)C1C(=O)CC(c2ccccc2)CC1=O] |
| (j) | ACOi-12-10 and ACOi-74-12-10 | 3-[3-(4-tert- butylphenyl)prop-2- enoyl]-6-methyl-3,4- dihydro-2H-pyran-2,4- dione | 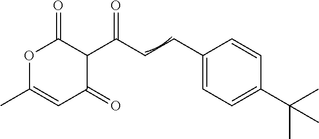 [SMILES: CC1=CC(=O)C(C(=O)CCc2ccc(C(C)(C)C)cc2)C(=O)O1] |
| (k) | ACOi-68-6-11 | 2-[(2E)-3-(furan-2- yl)prop-2-enoyl]-2,3- dihydro-1H-indene-1,3- dione | 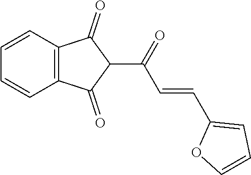 [SMILES: O=C(/C=C/c1ccco1)C1C(=O)c2ccccc2C1=O] |
| (l) | ACOi-74-12-12 | 3-[(2E)-3-[2- (difluoromethoxy)phenyl] prop-2-enoyl]-4- hydroxy-6-methyl-2H- pyran-2-one | 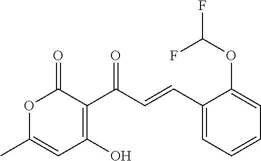 [SMILES: Cc1cc(O)c(C(=O)/C=C/c2ccccc2OC(F)F)c(=O)o1] |
| (m) | ACOi-74-12-14 | 3-[3-(4- ethylphenyl)prop-2- enoyl]-6-methyl-3,4- dihydro-2H-pyran-2,4- dione | 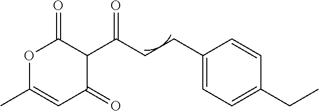 [SMILES: CCc1ccc(CCC(=O)C2C(=O)C=C(C)OC2=O)cc1] |
| (n) | ACOi-74-12-15 | 3-[3-(2,3- dichlorophenyl)prop-2- enoyl]-6-methyl-3,4- dihydro-2H-pyran-2,4- dione | 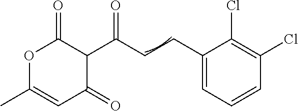 [SMILES: CC1=CC(=O)C(C(=O)CCc2cccc(Cl)c2Cl)C(=O)O1] |
| (o) | ACOi-74-12-16 | 3-[(2E)-3-(4- fluorophenyl)prop-2- enoyl]-4-hydroxy-6- methyl-2H-pyran-2-one | 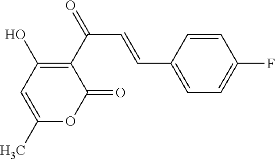 [SMILES: Cc1cc(O)c(C(=O)/C=C/c2ccc(F)cc2)c(=O)o1] |
| (p) | ACOi-74-12-17 | 3-{3-[4- (dimethylamino)phenyl] prop-2-enoyl}-6-methyl- 3,4-dihydro-2H-pyran- 2,4-dione | 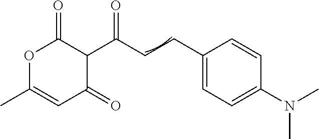 |
| (q) | ACOi-74-12-18 | 3-[3-(2,5- dimethylphenyl)prop-2- enoyl]-6-methyl-3,4- dihydro-2H-pyran-2,4- dione | 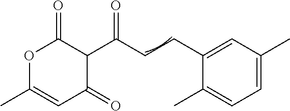 |
| (r) | ACOi-0-0-19 | 2-(3-oxo-3- phenylpropyl)cyclopentan- 1-one | 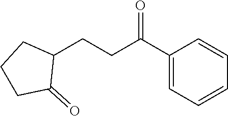 [SMILES: O=C(CCC1CCCC1=O)c1ccccc1] |
| (s) | ACOi-74-12-20 | 3-{3-[3-(4- chlorophenoxy)phenyl] prop-2-enoyl}-6-methyl- 3,4-dihydro-2H-pyran- 2,4-dione | 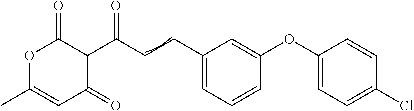 [SMILES: CC1=CC(=O)C(C(=O)CCc2cccc(Oc3ccc(Cl)cc3)c2)C(=O)O1] |
| (t) | ACOi-74-12-22 | 3-[3-(3- fluorophenyl)prop-2- enoyl]-6-methyl-3,4- dihydro-2H-pyran-2,4- dione | 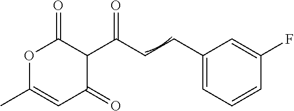 [SMILES: CC1=CC(=O)C(C(=O)CCc2cccc(F)c2)C(=O)O1] |
| (u) | ACOi-74-12-23 | 3-[(2E)-3-[4- (difluoromethoxy)phenyl] prop-2-enoyl]-4- hydroxy-6-methyl-2H- pyran-2-one | 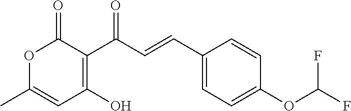 [SMILES: Cc1cc(O)c(C(=O)/C=C/c2ccc(OC(F)F)cc2)c(=O)o1] |

Preferably, the compound of the invention, or salt or tautomer thereof, is selected from (e), (g), (i), (l), (m), (n), (o), (t) and (u). These compounds may be advantageous because they are highly potent ACO inhibitors and therefore can be used in low doses to achieve effective inhibition. More preferably, the compound of the invention, or salt or tautomer thereof, may be compound (g) or compound (o).

Compounds (a) to (u) may be advantageous because they may be at least 10 fold, more preferably at least 50 fold, more preferably at least 100 fold, or most preferably at least 200 fold more potent than known ethylene response inhibitors such as AIB.

According to a second aspect of the invention there is provided a use of a compound of formula (1) for modifying at least one physiological process of a plant or plant part selected from:
a) preventing or slowing food ripening or crop maturation;
b) preventing or slowing plant or plant part senescence;
c) preventing or slowing flower senescence;
d) improving crop quality whilst on the plant or following harvest;
e) reducing an abiotic stress response in a plant, for example a response to heat and drought stress; and
f) maintaining the freshness of plants or plant parts; and wherein the compound of formula (1) is according to the first aspect of the invention.

Physiological processes (a) to (f) are induced by ethylene in plants after germination.

In certain embodiments, the invention provides uses of the above and below mentioned compounds in regulating stress responses in plants, including response to heat and drought stress as a likely impact of global warming, and reducing susceptibility to infection. These compounds are thus useful in protecting plants and especially field crops against a range of stresses found in extended periods of extreme temperatures (hot or cold), frost, pollution, wind, drought, flood, salt-stress, metal-stress, nutrient-stress, ozone levels, fungal infection, bacterial infection, exposure to bacterial pathogens or other stresses or any combination of stresses, to improve plant health, yield, vigour, or yield quality or volume, or the levels of defined natural products in plants, or other characteristics. The methods are thus applicable to any type of environmental stress that a plant may experience, including both biotic and abiotic stresses. Thus, the invention provides uses of the compounds to increase the quality and/or yield of a plant crop, through blocking plant stress response inducing ethylene production, thereby avoiding adverse effects on plant growth, development and productivity and plant products yield, quality, and other characteristics. In some embodiments the invention provides use of the compounds to improve recovery of a plant or plant part from an induced stress response.

In preferred embodiments the compound of formula (1) is 5,5-dimethyl-2-(2-phenylacetyl)cyclohexane-1,3-dione of the formula:

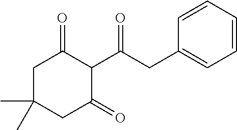 [SMILES: CC1(C)CC(=O)C(C(=O)Cc2ccccc2)C(=O)C1]

Within this document, 5,5-dimethyl-2-(2-phenylacetyl) cyclohexane-1,3-dione may also be referred to as ACOi-84-16-4 or 4B.

In alternative preferred embodiments the compound of formula (1) is 3-[(2E)-3-(4-fluorophenyl)prop-2-enoyl]-4-hydroxy-6-methyl-2H-pyran-2-one of the formula:

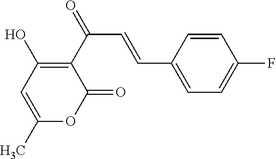 [SMILES: Cc1cc(O)c(C(=O)/C=C/c2ccc(F)cc2)c(=O)o1]

Within this document, 3-[(2E)-3-(4-fluorophenyl)prop-2-enoyl]-4-hydroxy-6-methyl-2H-pyran-2-one may also be referred to as ACOi-74-12-16 or 16B.

The compound formula (1) may be 2,2,4-trimethyl-6-(3-phenylpropanoyl)cyclohexane-1,3,5-trione, also known as Myrigalone A (MyA)

The compound of formula (1) may not be 2,2,4-trimethyl-6-(3-phenylpropanoyl) cyclohexane-1,3,5-trione, also known as Myrigalone A (MyA)

According to a third aspect of the invention there is provided a use of 5,5-dimethyl-2-(2-phenylacetyl)cyclohexane-1,3-dione for modifying at least one physiological process of a plant or plant part selected from the group consisting of:
a) preventing or slowing food ripening or crop maturation;
b) preventing or slowing plant or plant part senescence;
c) preventing or slowing flower senescence;
d) improving crop quality whilst on the plant or following harvest;
e) reducing a biotic or an abiotic stress response in a plant, for example a response to heat and drought stress;
f) maintaining the freshness of plants or plant parts;
g) preventing seed germination; and
h) weed control.

Physiological processes (a) to (h) are induced by ethylene in plants.

In preferred embodiments there is provided a use of 5,5-dimethyl-2-(2-phenylacetyl) cyclohexane-1,3-dione for preventing or slowing food ripening or crop maturation. In further preferred embodiments there is provided a use of 5,5-dimethyl-2-(2-phenylacetyl)cyclohexane-1,3-dione for preventing or slowing plant or plant part senescence. In further preferred embodiments there is provided a use of 5,5-dimethyl-2-(2-phenylacetyl)cyclohexane-1,3-dione reducing a biotic or an abiotic stress response in a plant, for example a response to heat and drought stress. In further preferred embodiments there is provided a use of 5,5-dimethyl-2-(2-phenylacetyl)cyclohexane-1,3-dione for improving plant stress response recovery following exposure to a biotic or an abiotic stress.

According to a fourth aspect of the invention there is provided a use of 3-[(2E)-3-(4-fluorophenyl)prop-2-enoyl]-4-hydroxy-6-methyl-2H-pyran-2-one for modifying at least one physiological process of a plant or plant part selected from the group consisting of:
a) preventing or slowing food ripening or crop maturation;
b) preventing or slowing plant or plant part senescence;
c) preventing or slowing flower senescence;
d) improving crop quality whilst on the plant or following harvest;
e) reducing a biotic or an abiotic stress response in a plant, for example a response to heat and drought stress;
f) maintaining the freshness of plants or plant parts;
g) preventing seed germination; and
h) weed control.

In preferred embodiments there is provided a use of 3-[(2E)-3-(4-fluorophenyl)prop-2-enoyl]-4-hydroxy-6-methyl-2H-pyran-2-one for preventing or slowing food ripening or crop maturation. In further preferred embodiments there is provided a use of 3-[(2E)-3-(4-fluorophenyl)prop-2-enoyl]-4-hydroxy-6-methyl-2H-pyran-2-one for preventing or slowing plant or plant part senescence. In further preferred embodiments there is provided a use of 3-[(2E)-3-(4-fluorophenyl)prop-2-enoyl]-4-hydroxy-6-methyl-2H-pyran-2-one reducing an abiotic stress response in a plant, for example a response to heat and drought stress. In further preferred embodiments there is provided a use of 3-[(2E)-3-(4-fluorophenyl)prop-2-enoyl]-4-hydroxy-6-methyl-2H-pyran-2-one for improving plant stress response recovery following exposure to a biotic or an abiotic stress.

According to a fifth aspect of the invention there is provided a method of inhibiting a post-germination ethylene production response of a plant or plant part comprising delivering a compound of formula (1) to a plant or plant part wherein the compound of formula (1) is according to the first aspect of the invention.

The plant part may be selected from the group consisting of: a leaf, stem, flower, seed, fruit or any combination thereof.

The following statements apply to any one of the first to fifth aspects of the invention. The compound of formula (1) may be delivered by spray. The compound of formula (1) may be delivered through watering. The compound of formula (1) may be provided as an aerosol, granules or an encapsulated form. The compound of formula (1) may be provided a liquid formulation. The compound of formula (1) may be provided as a form selected from the group consisting of: a solution, a suspension, an emulsion, a wettable powder, a soluble powder, water dispersible granules and water soluble bags or sachets.

According to a sixth aspect of the invention there is provided a method of modifying at least one physiological process of a plant or plant part selected from the group consisting of:
a) preventing or slowing the food ripening or crop maturation;
b) preventing or slowing plant or plant part senescence;
c) preventing or slowing flower senescence;
d) improving crop quality whilst on the plant or following harvest;
e) reducing a biotic or an abiotic stress response in a plant including a response to heat and drought stress; and
f) maintaining the freshness of a plant or plant part; and wherein the method comprises delivering a compound of formula (1) to the plant or plant part.

The compound of formula (1) may be according to the first aspect of the invention. The compound of formula (1) may be delivered according to the fifth aspect of the invention. The plant part may be selected from the group consisting of: a leaf, stem, flower, seed, fruit or any combination thereof.

In some embodiments the compound of formula (1) may be applied to a medium in which the plant or plant part is located, for subsequent uptake of the compound into the plant or plant part. The medium may be a liquid. The medium may comprise an aqueous solution. The medium may be an aqueous solution. The medium may comprise DMSO. The medium may be a solid. The medium may comprise any one or more of the group consisting of: soil, beads, an aqueous medium, a non-aqueous medium and water.

The compound of formula (1) may be directly applied to the plant or plant part. The compound of formula (1) may be directly applied to the plant or plant part as a solid or as a solution. The solution may be an aqueous solution.

The compound of formula (1) may be provided as a sole ingredient. The compound of formula (1) may be provided as an agrochemical composition. The agrochemical composition may comprise other agrochemicals. The agrochemical composition may comprise at least one ingredient selected from the group consisting of: a diluent, carrier, adjuvant and any combination thereof. The diluent or carrier may comprise at least one solvent. The diluent or carrier may comprise an aqueous medium. The diluent or carrier may comprise a hydrophobic diluent or carrier. The diluent or carrier may be selected from the group consisting of: an oil or a fat, a natural wax, a petroleum wax, a hydrocarbon, or any combination thereof. The adjuvant may be selected from the group consisting of: surfactants, crop oils, crop oil concentrates (COCs), vegetable oils, methylated seed oils (MSOs), petroleum oils, and silicone derivatives and any combination thereof.

In some embodiments the concentration of the compound of formula (1) in a medium or composition is between 0.005 μM and 50 μM. In some embodiments the concentration of the compound of formula (1) is between 0.01 μM and 50 μM, 0.05 μM and 50 μM, 1 μM and 50 μM, 0.005 μM and 40 μM, 0.01 0.005 μM and 40 μM, 0.005 μM and 30 μM, 0.01 μM and 30 μM, 0.005 M and 20 μM, 0.01 μM and 20 μM, 1 μM and 20 μM, 5 M and 20 μM, 0.005 μM and 15 μM, 0.01 μM and 15 μM, 0.05 μM and 15 μM, 0.1 μM and 15 μM, 1 μM and 15 μM, 5 μM and 15 μM or between 8 μM and 12 μM.

In some embodiments the compound of formula (1) is not 2,2,4-trimethyl-6-(3-phenylpropanoyl) yclohexane-1,3,5-trione.

In some embodiments the compound of formula (1) is 2,2,4-trimethyl-6-(3-phenylpropanoyl)cyclohexane-1,3,5-trione.

In some embodiments the compound of formula (1) is 5,5-dimethyl-2-(2-phenylacetyl) cyclohexane-1,3-dione.

In some embodiments the compound of formula (1) is 3-[(2E)-3-(4-fluorophenyl)prop-2-enoyl]-4-hydroxy-6-methyl-2H-pyran-2-one.

### DETAILED DESCRIPTION OF THE INVENTION

In order that the invention may be more clearly understood embodiments thereof will now be described, by way of example only, with reference to the accompanying drawings, of which:

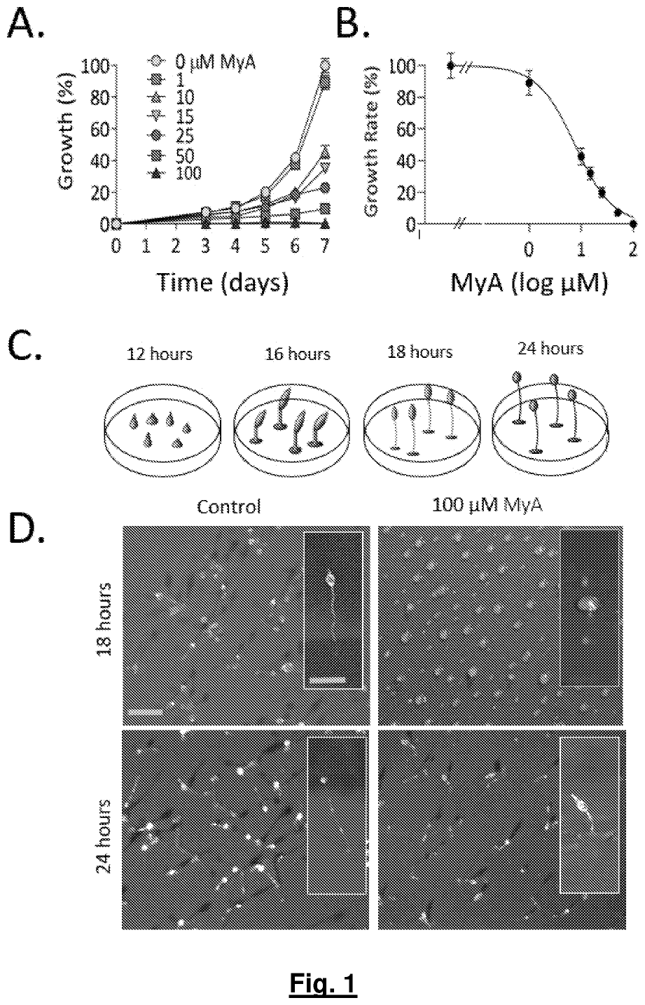

FIG. 1 (A) is a graph of the growth of D. discoideum on exposure to MyA at difference concentrations; (B) is a graph of the normalised growth rate of D. discoideum on exposure to MyA; (C) illustrates a schematic representation of wild type developmental phenotypes under control conditions, showing different stages of development; (D) illustrates developmental phenotypes, under control conditions, showing fruiting body morphology at 18 and 24 hours, from top down view and individual fruiting bodies in the absence of MyA (control) and in the presence of 100 μM of MyA.

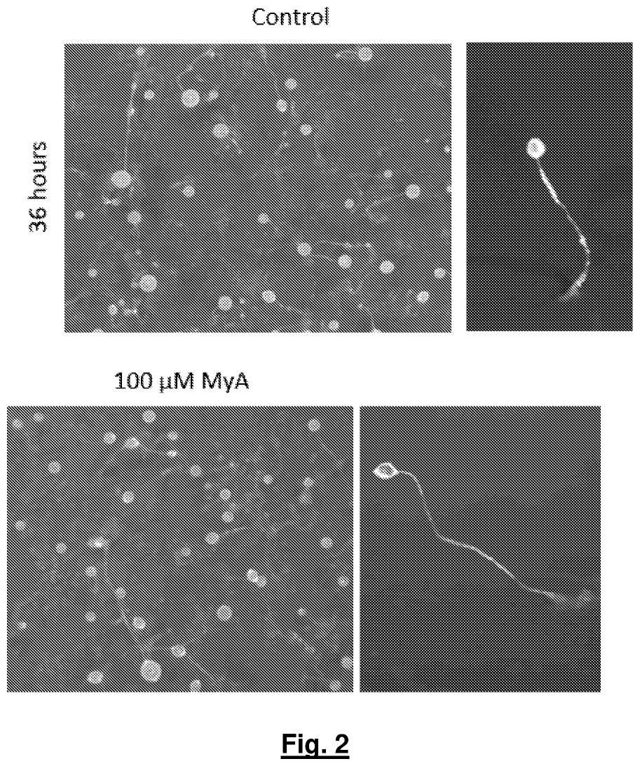

FIG. 2 illustrates the development of wild type D. discoideum after 36 hours in the absence of MyA (control) and in the presence of 100 μM of MyA.

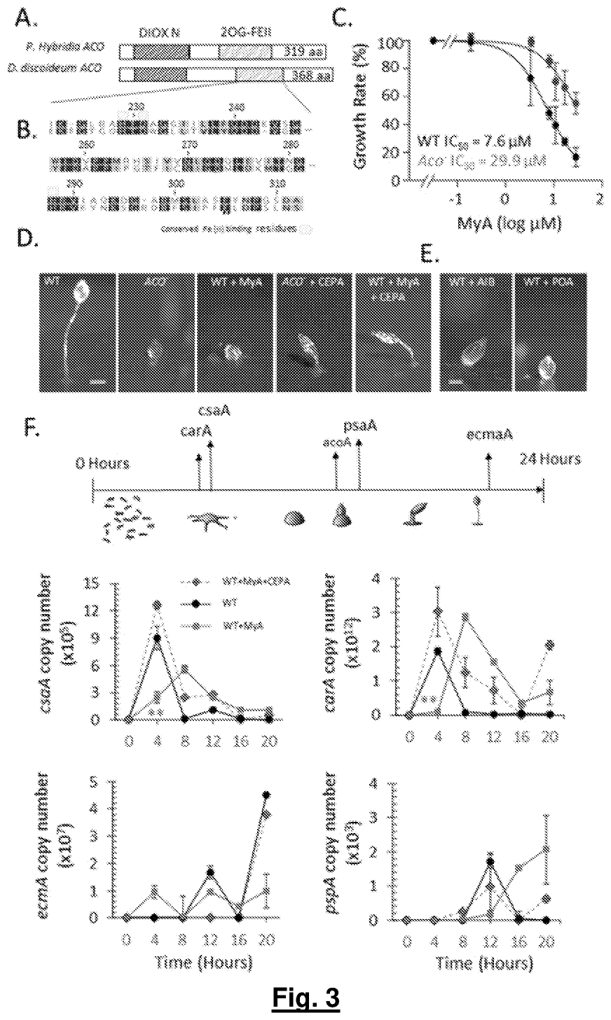

FIG. 3 (A) illustrates the common size and domain structure of the D. discoideum (ACO) and Petunia hybrida (ACO) proteins; (B) illustrates the conserved catalytic residues necessary for Fe (II) binding, consistent with orthologous function; (C) is a graph of the growth sensitivity of wild type and ACO-mutant cells in the presence of MyA; (D) illustrates wild type and ACO-mutant D. discoideum cell development at 20 h in the presence of MyA and/or CEPA; (E) illustrates wild type and ACO-mutant D. discoideum cell development at 20 h in the presence of AIB (10 μM) or POA (50 μM); (F) is a schematic of the developmental programme of D. discoideum on expression of specific developmental genes including csA (Contact site A), cAR1 (cAMP receptor 1), pspA (prespore-specific protein A), and ecmA (extracellular matrix protein A) and the graphs to show the absolute copy number of the genes in the absence of MyA, on exposure to MyA (100 μM) and on exposure to MyA and CEPA.

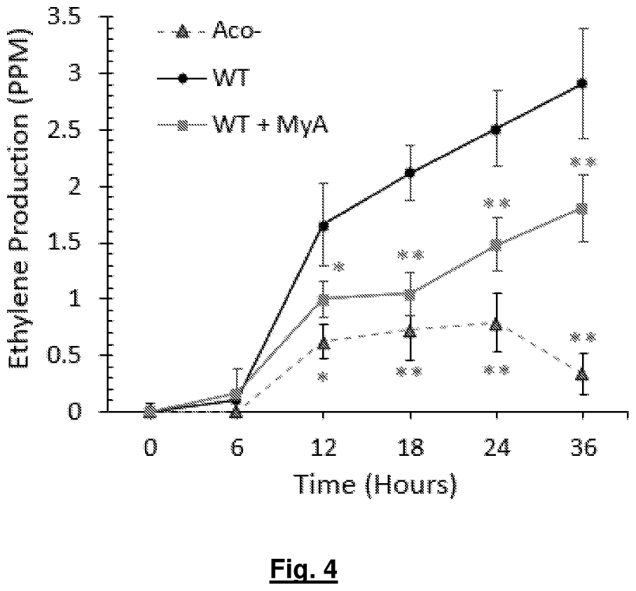

FIG. 4 is a graph which illustrates the wild type and ACO-mutant D. discoideum cell ethylene production over time in the absence and the presence of MyA.

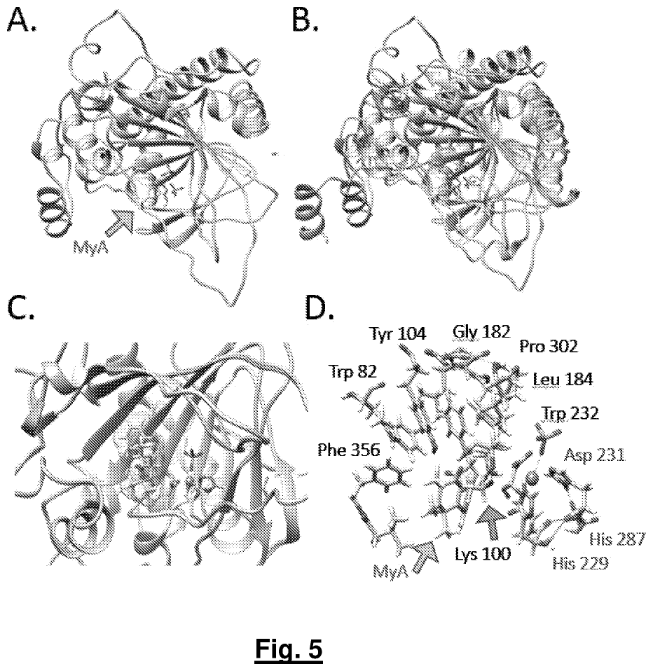

FIG. 5 illustrates modelled structures of D. discoideum ACO protein wherein A shows the MyA bonded to the ACO protein, B shows (commonality of D. discoideum and Petunia (plant ACO protein in purple)) structure, C shows binding of MyA adjacent to the catalytic site, and D close up of binding.

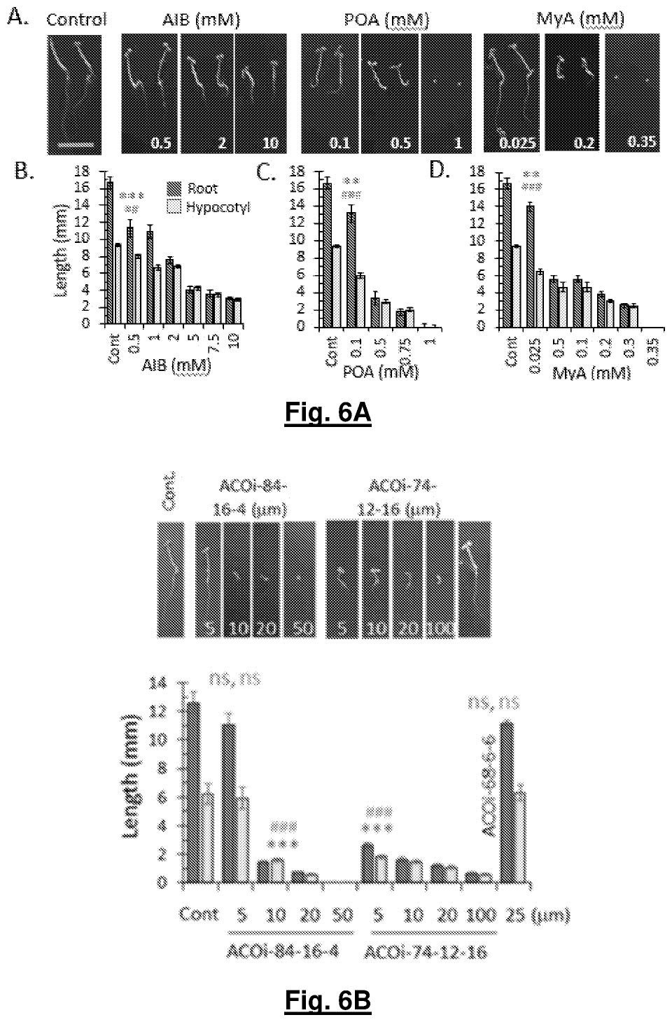

FIG. 6A illustrates A. thaliana crop maturation, root growth and hypocotyl growth after 6 days following exposure to MyA and also exposure to ethylene response inhibitors AIB and POA at higher concentrations.

FIG. 6B illustrates and also exposure to A. thaliana crop maturation, root growth and hypocotyl growth after 6 days following exposure to 5,5-dimethyl-2-(2-phenylacetyl)cyclohexane-1,3-dione (ACOi-84-16-4 or 4B) and 3-[(2E)-3-(4-fluorophenyl)rop-2-enoyl]-4-hydroxy-6-methyl-2H-pyran-2-one (ACOi-74-12-16 or 16B).

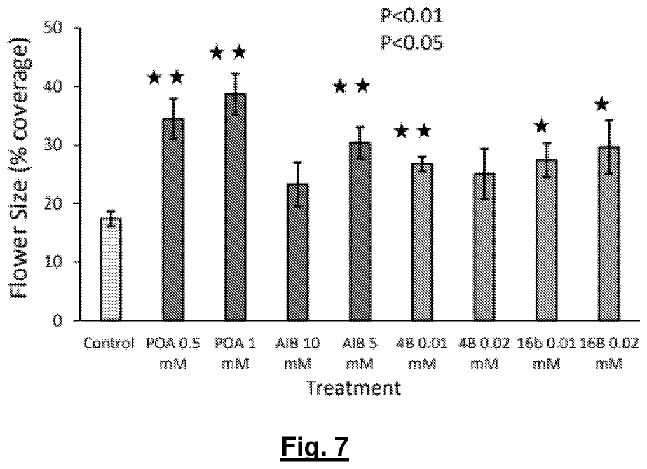

FIG. 7 a graph depicting the flower size of carnations after 9 days and treatment with no compound (control), POA, AIB, 5,5-dimethyl-2-(2-phenylacetyl)cyclohexane-1,3-dione (4B) or 3-[(2E)-3-(4-fluorophenyl)prop-2-enoyl]-4-hydroxy-6-methyl-2H-pyran-2-one (16B).

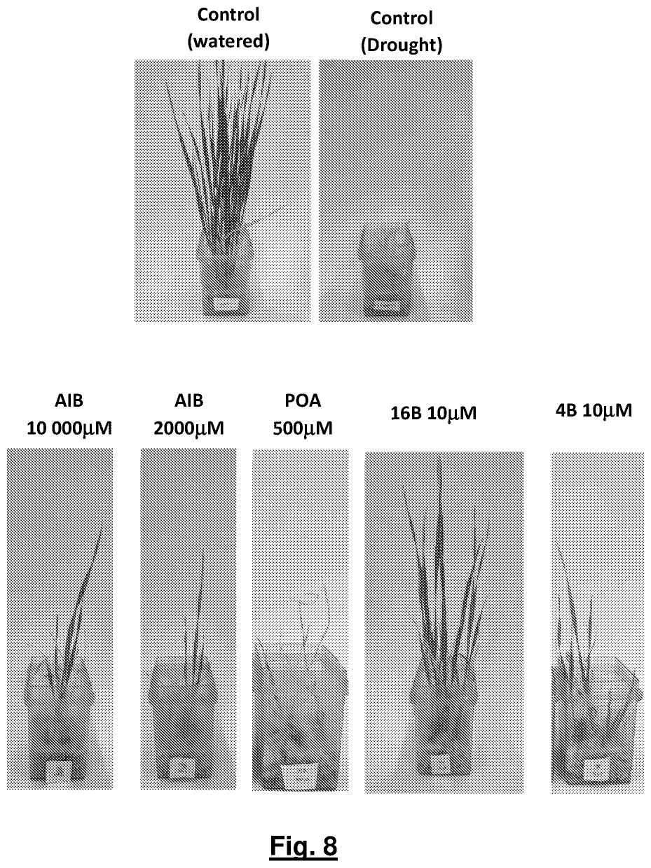

FIG. 8 End of test photos of barley plants following watering (control watered) or drought conditions in the absence of a treatment compound (control drought) or in the presence of AIB, POA or 3-[(2E)-3-(4-fluorophenyl)prop-2-enoyl]-4-hydroxy-6-methyl-2H-pyran-2-one (16B) or 5,5-dimethyl-2-(2-phenylacetyl)cyclohexane-1,3-dione (4B).

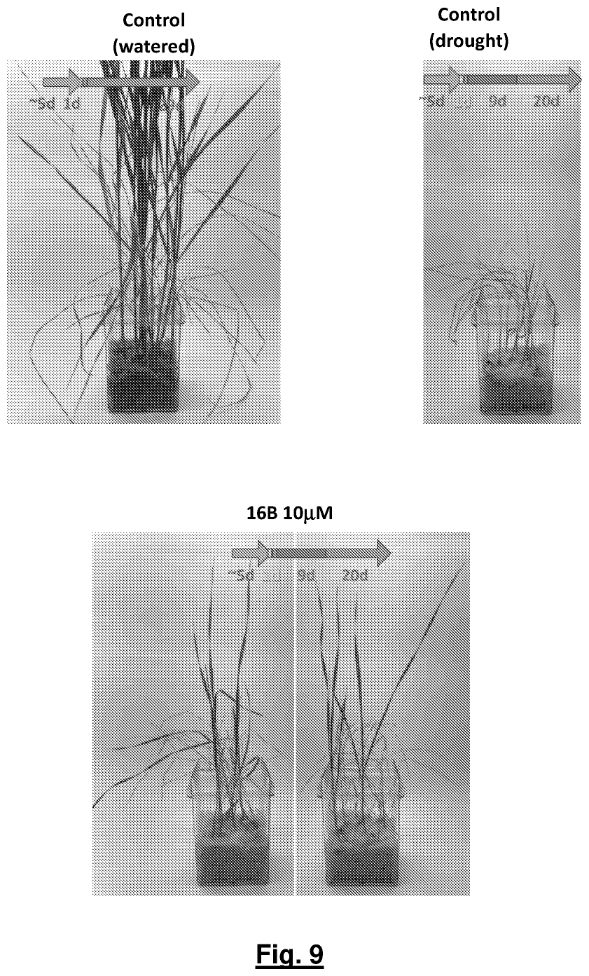

FIG. 9 End of test photos of wheat plants following watering (control watered) or drought conditions in the absence of a treatment compound (control drought) or in the presence of AIB or 3-[(2E)-3-(4-fluorophenyl)prop-2-enoyl]-4-hydroxy-6-methyl-2H-pyran-2-one (16B).

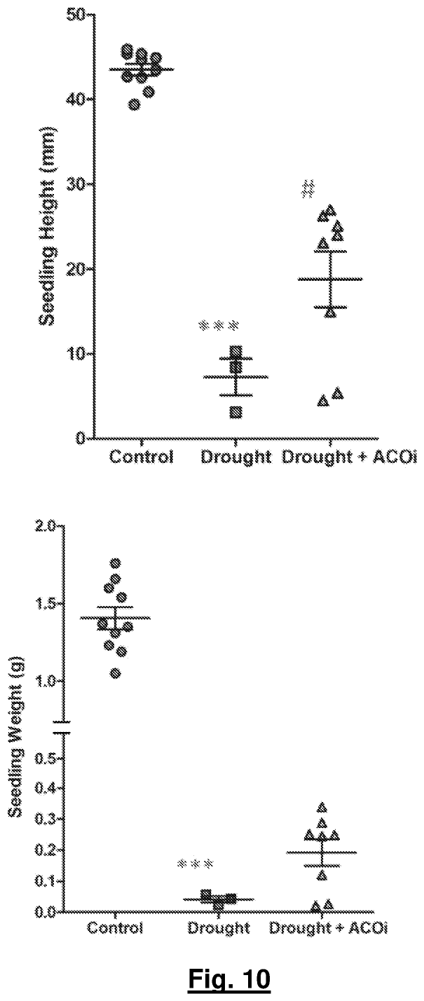

FIG. 10 a graph displaying the plant height of the plants of FIG. 9 wherein ACOi is 3-[(2E)-3-(4-fluorophenyl)prop-2-enoyl]-4-hydroxy-6-methyl-2H-pyran-2-one.

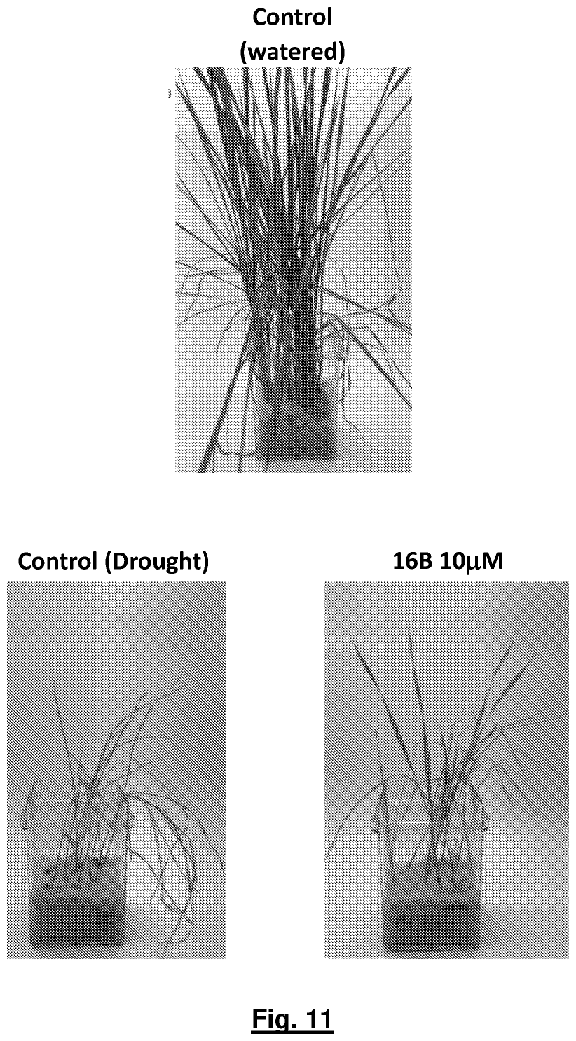

FIG. 11 End of test photos of rye plants following watering (control watered) or drought conditions in the absence of a treatment compound (control drought) or in the presence of 3-[(2E)-3-(4-fluorophenyl)prop-2-enoyl]-4-hydroxy-6-methyl-2H-pyran-2-one (16B) and following 19 days recovery post drought.

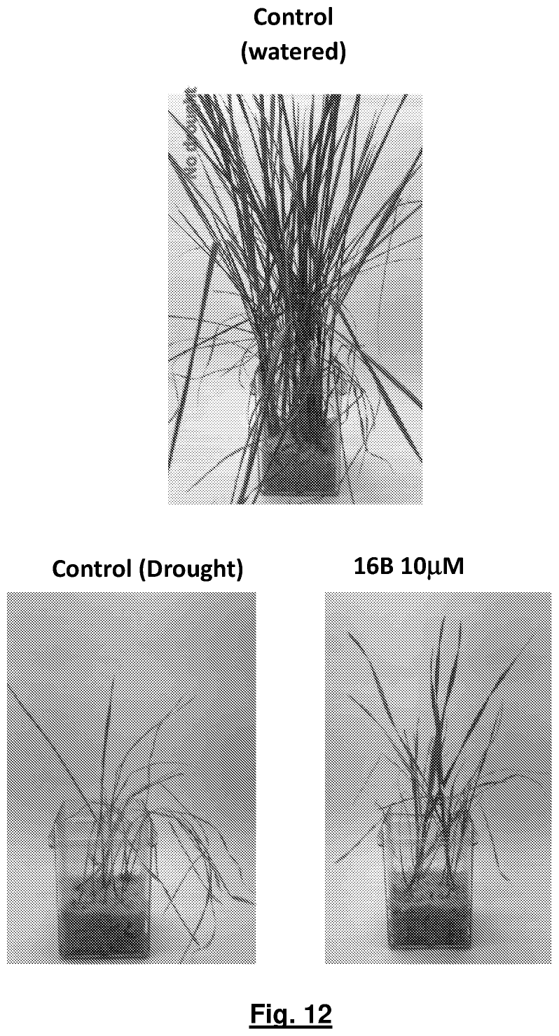

FIG. 12 End of test photos of rye plants following watering (control watered) or drought conditions in the absence of a treatment compound (control drought) or in the presence of 3-[(2E)-3-(4-fluorophenyl)prop-2-enoyl]-4-hydroxy-6-methyl-2H-pyran-2-one (16B) and following 23 days recovery post drought.

A first embodiment of a use of a compound of formula (1) for inhibiting a post-germination ethylene production response in a plant or plant part is provided wherein the compound of formula (1) is 2,2,4-trimethyl-6-(3-phenylpropanoyl) cyclohexane-1,3,5-trione or Myrigalone A (MyA) which is of the formula:

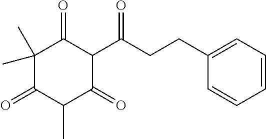 [SMILES: CC1C(=O)C(C(=O)CCc2ccccc2)C(=O)C(C)(C)C1=O]

Inhibition of D. discoideum Growth

The unicellular growth rate and multicellular of D. discoideum cells on exposure to MyA was tested and the results are illustrated in FIG. 1 and FIG. 2.

D. discoideum cells were divided by binary fission in nutrient-rich media, initially with a lag phase (0-120 h), and then an exponential phase. The D. discoideum cells were then exposed to MyA at concentrations of 0 μm, 1 μm, 10 μm, 15 μm, 25 μm, 50 μm and 100 μm. FIG. 1A illustrates that the MyA treatment caused a concentration-dependant inhibition of unicellular growth with a significant reduction at 10 μM (P<0.05) and a block in growth at 100 μM. FIG. 1B illustrates secondary plot analysis whilst provided an IC50 of 7.6 μM which shows that MyA is highly potent. This data demonstrates that D. discoideum growth is sensitive to the presence of low concentrations of MyA. This is advantageous because it shows that the effect of MyA on the model is potent.

The effect of MyA on multicellular development of D. discoideum was measured. The MyA was tested at a concentration of 100 μM. The control D. discoideum cells aggregated and differentiated over a 24-hour period to form a multicellular fruiting body consisting of a spore head, a stalk and a basal disk (FIG. 1C and 1D). 100 μM of MyA resulted in blocked cell growth and later stages of development were delayed.

FIG. 2 shows that after extended incubation (36 hours), MyA-treated cells developed into fully mature fruiting bodies, showing morphology similar to untreated cells. These results therefore suggest that high dose MyA is not lethal, but instead triggers a specific delay in development, likely caused by inhibition of molecular targets required for development rather than a generalized toxic effect. The data therefore shows a likely biochemical mechanism in the model to slow development, rather than a toxic effect to kill cells.

### Impact of MyA on Ethylene Production Response

The ACO enzyme functions as the rate limiting step in ethylene synthesis in plants, which is required for the release of seed dormancy and plant growth. The D. discoideum ACO protein was identified in a genetic resistance screen as a potential target for MyA, and bioinformatics analysis determined that the protein is a likely ortholog of the plant ACO protein. FIG. 3A and 3B illustrate that both proteins are of similar size (319 and 368 aa) and contain a common domain structure, with a conserved 2-oxoglutalate (2OG) and Fe (II) dependent oxygenase superfamily domains, necessary for the oxidation of organic substrates such as ACC, and conserved Fe (II) binding residues required for enzyme function. These characteristics provide evidence that the D. discoideum ACO protein is a homologue of the plant ACO protein, and thus functions in ethylene synthesis in D. discoideum.

The cell and developmental role of the D. discoideum ACO protein were investigated. FIG. 3C illustrates the resistance to the growth and developmental inhibitory effect of MyA on wild type D. discoideum cells (WT) and ACO ablated D. discoideum cells (ACO-). The results show around a four-fold reduction in potency in the ACO-mutant (IC50 of 29.9 μM) on exposure to MyA. This result is consistent with a loss of ACO protein as a primary target for MyA, where loss of the enzyme reduces sensitivity to growth inhibition.

The effect of ACO loss on multicellular development was assessed. ACO-D. discoideum mutants were engineered and compared to standard wild type D. discoideum cells. FIG. 3D shows the multicellular development of the ACO-D. discoideum mutant (ACO-), the standard wild type D. discoideum cell (WT) and the impact of the exposure of 100 μM of MyA. ACO-mutant showed a block in multicellular development at the mound stage, after around 12 hours of development, suggesting a delay of around 6 hours. Interestingly, this developmental defect was identical to that shown in the treatment of wild type cells with MyA (100 μM). To investigate this further, the development assays were repeated in the presence of exogenous ethylene, provided through the breakdown of 2-chloroethylphosphonic acid (CEPA) the results (ACO-+CEPA and WT+MyA+CEPA) showed that the developmental delay caused by both MyA treatment and ACO loss were partially rescued by the addition of ethylene (FIG. 3D).

A similar development delay is also evident following treatment with two structurally distinct ACO inhibitors, AIB (2-amino oxyisobutyric acid) and POA (pyrazinecarboxylic acid) (FIG. 3E), and an inhibitor of the plant ethylene receptors 1-methylcyclopropene.

Without being bound by theory, it is understood that this data shows that the D. discoideum protein is a functional ACO enzyme, that ethylene production is necessary for timely late development, and that the bioactivity of MyA in D. discoideum development is through the ACO inhibition to block ethylene production.

To provide quantitative analysis of the developmental effects of MyA, wild type cells were induced to develop on nitrocellulose filters for time periods between 0 and 20 hours, using cells under solvent only conditions, in the presence of MyA (100 μM), or in the presence of MyA and CEPA. The results are shown in FIG. 3F. Developmental gene expression associated with early aggregation (csA), cAR1 mid development (PspA), and in late development (ecmA) were assessed using qPCR. Analysis of csA expression showed peak expression levels at around 4 hours in untreated wild type cells that was delayed with MyA treatment rescued by exogenous ethylene. Similar delays in peak gene expression were also seen for carA (peak 4 h), and pspA (peak 12 h), and ecmA (peak 20 h), following MyA treatment, and these delays were rescued by addition of exogenous ethylene. These experiments confirm a MyA-dependent delay in D. discoideum development that is rescued by exogenous ethylene application, consistent with a role for MyA in inhibiting ACO activity and slowing crop maturation.

Wild type cells in the presence or absence of MyA (500 μM), or ACO-cells were maintained in sealed small flasks with limited head space over 36 h, and headspace gas was taken at 6-hour intervals and analysed by GCMS. The results are shown in FIG. 4. FIG. 4 shows that wild type cells showed increasing ethylene production after 6 hours, and levels increased more slowly up to 36 hours. MyA treatment significantly reduced ethylene production, causing a 39.5% decrease in ethylene production at 12 hours (P=0.031) which was maintained up to 36 hours (37.9% decrease, P=0.0025). Similarly, ACO-cells also showed a significantly reduced level of ethylene after 12 hours of 62.4% (p=0.0045) that remained low throughout the analysis. These findings show that D. discoideum cells produce ethylene during starvation, and ethylene production is reduced in the presence of MyA, consistent with the inhibition of ACO in the ethylene synthesis pathway.

Direct Binding of MyA to ACO To identify a potential direct mechanism of MyA dependent ACO inhibition, a range of molecular modelling techniques were used. The tertiary structure of D. discoideum ACO protein was predicted using phyre2 based upon the closest available crystal structure (Petunia ACO: PDB:5LUN) as a template (FIG. 5A). The D. discoideum ACO protein and P. hybridia ACO protein are predicted to share a common structure, featuring a double-stranded-helix jellyroll fold surrounded by alpha-helices, with superimposed structures provide a root-mean-square deviation of 1.016 angstroms over 282 aligned CA atoms (FIG. 5B). This analysis identified high 3D structural conservation between the key Fe²⁺ dioxygenase domain required for catalytic activity between both proteins. It is understood that despite a lack of crystal structure binding analysis, the ACO substrate, 1-aminocyclopropane-1-carboxylic acid (ACC) is likely to bind directly to Fe²⁺ via its carboxylate and amino groups during catalysis, and thus substrate access to the facial triad is essential for ACO activity. It was shown, via predictive docking assays, that MyA binds within the binding pocket of the facial triad, and via a hydrogen bonding to Lys 100 (FIG. 5C and 5D).

### Preventing or Slowing Crop Maturation

A first embodiment of the use of a compound of formula (1) for modifying a physiological process of a plant or plant part according to the second aspect of the invention was provided wherein the compound of formula (1) is 2,2,4-trimethyl-6-(3-phenylpropanoyl)cyclohexane-1,3,5-trione or Myrigalone A (MyA) and the physiological process was preventing or slowing crop maturation.

A second embodiment of the use of a compound of formula (1) for modifying a physiological process of a plant or plant part according to the second aspect of the invention was provided wherein the compound of formula (1) is 5,5-dimethyl-2-(2-phenylacetyl)cyclohexane-1,3-dione and the physiological process was preventing or slowing crop maturation.

The formula of 5,5-dimethyl-2-(2-phenylacetyl) cyclohexane-1,3-dione is:

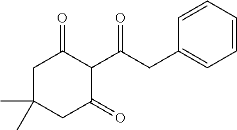 [SMILES: CC1(C)CC(=O)C(C(=O)Cc2ccccc2)C(=O)C1]

Within this document, 5,5-dimethyl-2-(2-phenylacetyl) cyclohexane-1,3-dione may also be referred to as ACOi-84-16-4 or 4B.

A third embodiment of the use of a compound of formula (1) for modifying a physiological process of a plant or plant part according to the second aspect of the invention was provided wherein the compound of formula (1) is 3-[(2E)-3-(4-fluorophenyl)prop-2-enoyl]-4-hydroxy-6-methyl-2H-pyran-2-one and the physiological process was preventing or slowing crop maturation.

The formula of 3-[(2E)-3-(4-fluorophenyl)prop-2-enoyl]-4-hydroxy-6-methyl-2H-pyran-2-one is:

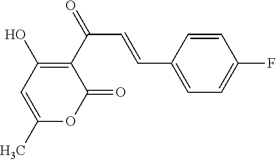 [SMILES: Cc1cc(O)c(C(=O)/C=C/c2ccc(F)cc2)c(=O)o1]

Within this document, 3-[(2E)-3-(4-fluorophenyl)prop-2-enoyl]-4-hydroxy-6-methyl-2H-pyran-2-one may also be referred to as ACOi-74-12-16 or 16B.

A model illustrating the binding of MyA to the active site of plant ACO enzymes is illustrated in FIG. 5. 16 compounds were selected and tested for bioassay efficacy analysis in this model (100 μM) with the data presented in table 2.

TABLE 2

| Name | No. | Activity* | IUPAC Name |
|---|---|---|---|
| ACOi-57-1 | 1 | — | 1,3-dimethyl-5-[2-oxo-2-(piperidin-1-yl)ethyl]-1,3,5- |
| | | | triazinane-2,4,6-trione |
| ACOi-28-2 | 2 | — | N-[(4-chlorophenyl)methyl]-2,6-dimethoxybenzamide |
| ACOi-46-3 | 3 | — | (2E)-1-(2-hydroxy-4,6-dimethoxyphenyl)-3-phenylprop- |
| | | | 2-en-1-one |
| ACOi-21-4 | 4 | — | 1,3-dimethyl-5-(3-oxobutanoyl)-1,3-diazinane-2,4,6-trione |
| ACOi-70-5 | 5 | — | 2-(5,5-dimethyl-2,4-dioxo-1,3-oxazolidin-3-yl)-N- |
| | | | [(2-methoxyphenyl)methyl]acetamide |
| ACOi-68-6 | 6 | — | 2-[(2E)-3-(4-iodophenyl)prop-2-enoyl]-2,3-dihydro-1H- |
| | | | indene-1,3-dione |
| ACOi-77-7 | 7 | * | 5-benzoyl-4,5,6,7-tetrahydro-1-benzofuran-4-one |
| ACOi-67-8 | 8 | * | methyl 5-acetyl-2,2-dimethyl-4,6-dioxocyclohexane-1-carboxylate |
| ACOi-62-9 | 9 | — | 6-amino-1,3-dimethyl-5-{2-[(1-methyl-1H-1,2,3,4-tetrazol- |
| | | | 5-yl)-sulfanyl]acetyl}-1,2,3,4-tetrahydropyrimidine-2,4-dione |
| ACOi-69-10 | 10 | * | 6-amino-5-{2-[(3,4-dichlorophenyl)amino]acetyl}-1,3- |
| | | | dimethyl-1,2,3,4-tetrahydropyrimidine-2,4-dione |
| ACOi-54-11 | 11 | ** | 2,6-dimethoxyphenyl 3-(5-methylfuran-2-yl)prop-2-enoate |
| ACOi-74-12 | 12 | *** | 3-[3-(3-bromo-4-fluorophenyl)prop-2-enoyl]-6- |
| | | | methyl-3,4-dihydro-2H-pyran-2,4-dione |
| ACOi-72-13 | 13 | ND | 3-[3-(3-chlorophenyl)prop-2-enoyl]-6-methyl-3,4- |
| | | | dihydro-2H-pyran-2,4-dione |
| ACOi-25-14 | 14 | — | (2E)-3-(4-chlorophenyl)-1-(2,4,6-trimethoxyphenyl)prop-2-en-1-one |
| ACOi-39-15 | 15 | ND | (2E)-3-(2-chlorophenyl)-1-(2,4,6-trimethoxyphenyl)prop-2-en-1-one |
| ACOi-84-16 | 16 | *** | 5,5-dimethyl-2-(2-phenylacetyl)cyclohexane-1,3-dione |

Table 2 shows the effect of novel compounds on D. discoideum ACO-inhibition dependent development block at mound formation. In this assay, D. discoideum WT cells were starved on nitrocellulose filters at 100 μM of indicated compounds, incubated for 20 hours (22° C.), and developmental block at the mound stage was assessed, where-indicates no effect, * indicates some effect, ** indicates strong effect, and *** indicates potent effect similar to MyA, and ND not determined.

5,5-dimethyl-2-(2-phenylacetyl) cyclohexane-1,3-dione (ACOi-84-16-4) and 3-[(2E)-3-(4-fluorophenyl)prop-2-enoyl]-4-hydroxy-6-methyl-2H-pyran-2-one (ACOi-74-12-16) demonstrated potent efficacy in this model.

Since seed germination and root/hypocotyl extension of A. thaliana and other plant species are promoted by ACC and ethylene, a role for MyA in these processes was also investigated.

In these experiments, A. thaliana seeds were germinated and grown in 24 h light conditions for 6 days and root and hypocotyl length were recorded (FIG. 6A and 6B). FIG. 6A shows that both AIB and POA treatment provided a dose-dependent reduction in root and hypocotyl growth. MyA treatment produced a comparative reduction in both root and hypocotyl growth, but with greater potency than the established ACO inhibitors. In particular the lowest concentration of MyA which was shown to have an effect on growth (0.025 mM), was 20 times lower than the concentration of AIB, and 4 times lower than the concentration of POA.

FIG. 6B shows that 5,5-dimethyl-2-(2-phenylacetyl) cyclohexane-1,3-dione (ACOi-84-16-4) and 3-[(2E)-3-(4-fluorophenyl)prop-2-enoyl]-4-hydroxy-6-methyl-2H-pyran-2-one (ACOi-74-12-16) result in a dose dependent effect on root/hypocotyl growth from as low as 5 μm compared to the control which was only exposed to DMSO and compared to AIB and POA when used at higher concentrations.

The IC50 values for the root or hypocotyl extension after 6 days for 5,5-dimethyl-2-(2-phenylacetyl) cyclohexane-1,3-dione (ACOi-84-16-4), 3-[(2E)-3-(4-fluorophenyl)prop-2-enoyl]-4-hydroxy-6-methyl-2H-pyran-2-one (ACOi-74-12-16) and MyA, compared to known compounds AIB and POA are presented in table 3. Images of the plant growth and the root hairs are shown in FIG. 6B for 5,5-dimethyl-2-(2-phenylacetyl) cyclohexane-1,3-dione (ACOi-84-16-4) and 3-[(2E)-3-(4-fluorophenyl)prop-2-enoyl]-4-hydroxy-6-methyl-2H-pyran-2-one (ACOi-74-12-16) and FIG. 6A for MyA and comparably for AIB and POA.

TABLE 3

| | Root extension or | |
|---|---|---|
| Compound | hypocotyl extension | IC50/μM |
| Control - AIB | Root | 1617 |
| Control - AIB | Hypocotyl | 4293 |
| Control - POA | Root | 213 |
| Control - POA | Hypocotyl | 184 |
| MyA | Root | 53.3 |
| MyA | Hypocotyl | 59.4 |
| ACOi-84-16-4 | Root | 6.9 |
| ACOi-84-16-4 | Hypocotyl | 8.2 |
| ACOi-74-12-16 | Root | 1.02 |
| ACOi-74-12-16 | Hypocotyl | 0.79 |

The data in table 3 shows that the novel compounds of formula (1) (MyA, ACOi-84-16-4 and ACOi-74-12-16) result in a potent inhibition of hypocotyl or root extension wherein the potency of 5,5-dimethyl-2-(2-phenylacetyl) cyclohexane-1,3-dione (ACOi-84-16-4) and 3-[(2E)-3-(4-fluorophenyl)prop-2-enoyl]-4-hydroxy-6-methyl-2H-pyran-2-one is over 5400-fold or 1580-fold compared to AIB respectively. All three compounds of formula (1) result in a prevention or slowing of crop maturation. In particular 5,5-dimethyl-2-(2-phenylacetyl) cyclohexane-1,3-dione (ACOi-84-16-4) and 3-[(2E)-3-(4-fluorophenyl)prop-2-enoyl]-4-hydroxy-6-methyl-2H-pyran-2-one (ACOi-74-12-16) are highly potent and therefore are advantageous because they can be used at significantly lower concentrations.

### Preventing or Slowing Flower Senescence

A fourth embodiment of the use of a compound of formula (1) for modifying at least one physiological process of a plant or plant part according to the second aspect of the invention was provided wherein the compound of formula (1) is 5,5-dimethyl-2-(2-phenylacetyl)cyclohexane-1,3-dione and the physiological process was preventing or slowing flower senescence.

A fifth embodiment of the use of a compound of formula (1) for modifying at least one physiological process of a plant or plant part according to the second aspect of the invention was provided wherein the compound of formula (1) is 3-[(2E)-3-(4-fluorophenyl)prop-2-enoyl]-4-hydroxy-6-methyl-2H-pyran-2-one and the physiological process was preventing or slowing flower senescence.

The slowing of flower senescence in the presence of 5,5-dimethyl-2-(2-phenylacetyl)cyclohexane-1,3-dione or 3-[(2E)-3-(4-fluorophenyl)prop-2-enoyl]-4-hydroxy-6-methyl-2H-pyran-2-one was tested and compared to a control untreated sample and known ethylene production inhibitors POA and AIB.

Carnations were cut to approximately 5 cm long stems and placed in a solution wherein the solution comprised 5 mL water and either no additional components (control), 0.01 mM or 0.02 mM of 5,5-dimethyl-2-(2-phenylacetyl) cyclohexane-1,3-dione (labelled as 4B) or 3-[(2E)-3-(4-fluorophenyl)prop-2-enoyl]-4-hydroxy-6-methyl-2H-pyran-2-one (labelled as 16B) or 0.5 mM or 1 mM of POA or 10 mM or 5mM of AIB were added. The flower size coverage was measured and the petal shrinkage, or reduction in flower size, is used as an indicator of flower senescence. The results are shown in FIG. 7.

5,5-dimethyl-2-(2-phenylacetyl) cyclohexane-1,3-dione and 3-[(2E)-3-(4-fluorophenyl)prop-2-enoyl]-4-hydroxy-6-methyl-2H-pyran-2-one showed significant effect compared to the control sample showing that both compounds effectively slow flower senescence at low concentrations.

5,5-dimethyl-2-(2-phenylacetyl) cyclohexane-1,3-dione and 3-[(2E)-3-(4-fluorophenyl)prop-2-enoyl]-4-hydroxy-6-methyl-2H-pyran-2-one demonstrated comparable performance to significantly higher concentrations of known ethylene inhibitors POA and AIB thereby showing that they are more potent. This is advantageous because it means that the compounds can be used at much lower concentrations which may be cheaper for the consumer whilst successfully extending the lifetime of the cut flower compared to no treatment.

### Reducing a Drought Stress Response in a Plant

A sixth embodiment of the use of a compound of formula (1) for modifying at least one physiological process of a plant or plant part according to the second aspect of the invention was provided wherein the compound of formula (1) is 5,5-dimethyl-2-(2-phenylacetyl) cyclohexane-1,3-dione and the physiological process was reducing an abiotic stress response in a plant, to enhance stress recovery, wherein the abiotic stress response is a response to heat and drought stress.

A seventh embodiment of the use of a compound of formula (1) for modifying at least one physiological process of a plant or plant part according to the second aspect of the invention was provided wherein the compound of formula (1) is 3-[(2E)-3-(4-fluorophenyl)prop-2-enoyl]-4-hydroxy-6-methyl-2H-pyran-2-one and the physiological process was reducing an abiotic stress response in a plant, to enhance stress recovery, wherein the abiotic stress response is a response to heat and drought stress.

The reduction of drought stress in the presence of 5,5-dimethyl-2-(2-phenylacetyl) cyclohexane-1,3-dione or 3-[(2E)-3-(4-fluorophenyl)prop-2-enoyl]-4-hydroxy-6-methyl-2H-pyran-2-one was tested and compared to a control untreated sample.

The plants tested were barley, wheat and rye plants.

The seeds of each plant were washed with 5% sodium hypochlorite for 15 minutes (with shaking at 120 rpm) and then washed three times in sterile distilled water. The seeds were placed on two layers of waterlogged 3 MM for ˜72 hours under lights at RT until hypocotyl emerged. 7-10 germinated seeds were incubated in sterile Magenta boxes containing 100 mL perlite per box, moistened with 60 ml sterile distilled water and autoclaved. The seeds were incubated in a growth cabinet with a 12 hour light/dark cycle at 22° C. until the first seedling reached approximately 5 cm height. This took approximately 4-5 days. The water was then replaced with 0.5×Hoagland solution with the test compounds (or DMSO for control samples) for 24 hour (Hoagland's No2 Basal salt mixture, Sigma, H2395, use 1.6 g/L in sterile water and autoclaved), and returned to growth cabinet. The media was then removed, the perlite was rinsed twice with sterile distilled water and the solution was replace with 35 ml 0.5 ×Hoaglands solution and returned to the growth cabinet for 24 hours.

The no drought sample was then grown in 0.5×Hoaglands solution and the solution was topped up daily.

The drought samples were exposed to drought conditions for 9 days by removing the media and returned to the growth cabinet. At recovery, 0.5×Hoaglands solution was added back to the drought sample Magenta boxes, up to the top of the perlite and this level was maintained during recovery. The plant recovery over time (up to 19 days post rescue) was recorded and the photographs from the end of the experiment are illustrated in FIG. 8, FIG. 9 and FIG. 11.

10 μM of 3-[(2E)-3-(4-fluorophenyl)prop-2-enoyl]-4-hydroxy-6-methyl-2H-pyran-2-one (labelled as 16B), 10 μM of 5,5-dimethyl-2-(2-phenylacetyl) cyclohexane-1,3-dione (labelled as 4B) and 10,000 μM 2,000 μM of AIB and 500 μM of POA were tested on barley seedlings. The results after 5 days of growth, 9 days of drought and 12 days of normal watered conditions are shown in FIG. 8. The barley treated with 500 μM of POA did not show positive result as most of the seedlings died during the drought period and did not recover. The barley treated with 2,000 μM of AIB did slightly better and the barley did better still with 10,000 μM AIB however all samples treated with POA or AIB did not recover to the same seedling height as the watered control sample. The barley exposed to 10 μM of 3-[(2E)-3-(4-fluorophenyl)prop-2-enoyl]-4-hydroxy-6-methyl-2H-pyran-2-one showed comparable seedling height to the control sample which had been watered throughout the duration and therefore shows that 3-[(2E)-3-(4-fluorophenyl)prop-2-enoyl]-4-hydroxy-6-methyl-2H-pyran-2-one provides a significant benefit over known AIB or no compound in high stress, drought conditions. 3-[(2E)-3-(4-fluorophenyl)prop-2-enoyl]-4-hydroxy-6-methyl-2H-pyran-2-one also shows a high potency as it is shown to be effected at 10 μM. The barley exposed to 10 μM of 5,5-dimethyl-2-(2-phenylacetyl)cyclohexane-1,3-dione showed comparable growth to the same treated with 10,000 μM AIB showing that 5,5-dimethyl-2-(2-phenylacetyl)cyclohexane-1,3-dione is significantly more potent than known POA or known AIB. This data shows that barley seedlings treated with compounds of formula (1) recover better after periods of drought, or high stress, which is advantageous for growing crops in areas with variable weather conditions and improving the crop yield in those areas.

The drought testing was repeated with wheat seedlings and the results are shown in FIGS. 9 and 10 wherein the data was measured following 5 days of growth, 9 days of drought and 19 days of recovery. The ACOi treated sample was treated with 10 μM of 3-[(2E)-3-(4-fluorophenyl)prop-2-enoyl]-4-hydroxy-6-methyl-2H-pyran-2-one after the initial 5 days of growth. The wheat seedlings showed improved recovery upon treatment compared to the control drought sample which was not treated. This is shown by the increased height and mass of the wheat seedlings as shown in FIG. 10 wherein the control sample was not exposed to the drought, the drought sample showed the shortest seedling height and the lowest seedling mass, and the drought +ACOi samples showed improved seedling growth. This improved seedling growth upon treatment with 10 μM of 3-[(2E)-3-(4-fluorophenyl)prop-2-enoyl]-4-hydroxy-6-methyl-2H-pyran-2-one, which is a low concentration of a compound of formula (1), reflects better drought stress recovery, and is advantageous because it would lead to improved crop yield following treatment by reducing the stress response of the plant following periods of drought.

The drought testing was repeated with rye seedlings and the results are shown in FIG. 11 wherein the data was measured following 5 days of growth, 9 days of drought and 19 days of recovery. The ACOi treated sample was treated with 10 μM of 3-[(2E)-3-(4-fluorophenyl)prop-2-enoyl]-4-hydroxy-6-methyl-2H-pyran-2-one after the initial 5 days of growth. The rye seedlings showed improved recovery upon treatment compared to the control drought sample which was not treated. The recovery period was extended to 23 days. The photos of the control sample and the treated sample after 23 days recover is shown in FIG. 12. The ACOi treated sample continues to recover and results in a number of green leaves as the plant grows, and the drought sample showed the shortest seedling height. This improved seedling growth upon treatment with 10 μM of 3-[(2E)-3-(4-fluorophenyl)prop-2-enoyl]-4-hydroxy-6-methyl-2H-pyran-2-one, which is a low concentration of a compound of formula (1), reflects better drought stress recovery, and is advantageous because it would lead to improved crop yield following treatment by reducing the stress response of the plant following periods of drought.

The above testing demonstrates that the compounds 3-[(2E)-3-(4-fluorophenyl)prop-2-enoyl]-4-hydroxy-6-methyl-2H-pyran-2-one and 5,5-dimethyl-2-(2-phenylacetyl) cyclohexane-1,3-dione are suitable for reducing a post-germination abiotic stress response recovery in a plant, for example a response to heat and drought stress after germination, and can be used with a variety of plants, in particular a variety of crops. This is advantageous as changing climates are resulting in more countries experiencing drought periods and therefore crop shortages and therefore the compounds of formula (1) are advantageous to address this and improve consistency of crop supply in periods of unpredictable weather which may otherwise cause stress to the plant.

The above embodiments are described by way of example only. Many variations are possible without departing from the scope of the invention as defined in the appended claims.

1. A compound of formula (1), wherein formula (1) is: 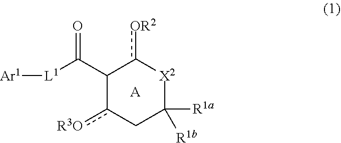
or a salt or tautomer thereof, wherein:
ring A is a six-membered aromatic or non-aromatic ring in which X¹ and X² are independently selected from O, CH, CH₂, CH(C₁₋₄ alkyl), and C(C₁₋₄ alkyl)₂;
R^{1a} and R^{1b} are independently selected from hydrogen and C₁₋₄ alkyl or R^{1a} and R^{1b} together form a carbonyl group with the carbon atom of ring A to which they are attached;
R² and R³ are independently selected from hydrogen and C₁₋₄ alkyl or are absent when the oxygen atom to which they are attached forms a carbonyl group with the carbon ring member of ring A;
L¹ is selected from a bond, —CH₂, —CH═CH— and —CH₂—CH₂—Ar¹ is a 5- or 6-membered carbocyclic or heterocyclic aromatic group optionally substituted by one or more substituents R⁴; and
R⁴ is selected from hydroxy, halogen, O—Ar², Hyd¹, O—Hyd¹, NH(Hyd¹) and N(Hyd¹)₂, wherein Hyd¹ is a C₁₋₄ hydrocarbon group optionally substituted with one or more fluorine atoms;
Ar² is a 5- or 6-membered carbocyclic or heterocyclic aromatic group;
wherein the compound is operative to inhibit a post-germination ethylene production response in a plant or in a plant part.

2. The compound of claim 1, wherein the Use of a compound is capable of modifying at least one physiological process of a plant or plant part selected from:
a) preventing or slowing food ripening or crop maturation;
b) preventing or slowing plant or plant part senescence;
c) preventing or slowing flower senescence;
d) improving crop quality whilst on the plant or following harvest;
e) reducing a biotic or an abiotic stress response in a plant, for example a response to heat and drought stress;
f) maintaining the freshness of plants or plant parts; and
wherein formula (1) is 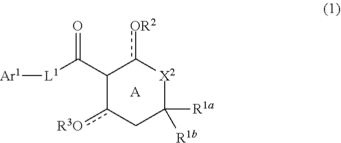
or a salt or tautomer thereof, wherein:
ring A is a six-membered aromatic or non-aromatic ring in which X¹ and X² are independently selected from O, CH, CH₂, CH(C₁₋₄ alkyl), and C(C₁₋₄ alkyl)₂;
R^{1a} and R^{1b} are independently selected from hydrogen and C₁₋₄ alkyl or R^{1a} and R¹b together form a carbonyl group with the carbon atom of ring A to which they are attached;
R² and R³ are independently selected from hydrogen and C₁₋₄ alkyl or are absent when the oxygen atom to which they are attached forms a carbonyl group with the carbon ring member of ring A;
L¹ is selected from a bond, —CH₂, —CH═CH— and —CH₂—CH₂—Ar¹ is a 5- or 6-membered carbocyclic or heterocyclic aromatic group optionally substituted by one or more substituents R⁴;
R⁴ is selected from hydroxy, halogen, O—Ar², Hyd¹, O—Hyd¹, NH(Hyd¹) and N(Hyd¹)₂, wherein Hyd¹ is a C₁₋₄ hydrocarbon group optionally substituted with one or more fluorine atoms;
Ar² is a 5- or 6-membered carbocyclic or heterocyclic aromatic group.

3. The compound according to claim 1, wherein the compound is not 2,2,4-trimethyl-6-(3-phenylpropanoyl) cyclohexane-1,3,5-trione.

4. The compound according to claim 1, wherein the compound is 5,5-dimethyl-2-(2-phenylacetyl) cyclohexane-1,3-dione.

5. The compound according to claim 1, wherein the compound is 3-[(2E)-3-(4-fluorophenyl)prop-2-enoyl]-4-hydroxy-6-methyl-2H-pyran-2-one.

6. The compound according to claim 1, wherein the compound is 5,5-dimethyl-2-(2-phenylacetyl) cyclohexane-1,3-dione, and wherein the compound is operative for modifying at least one physiological process of a plant or plant part selected from:
a) preventing or slowing food ripening or crop maturation;
b) preventing or slowing plant or plant part senescence;
c) preventing or slowing flower senescence;
d) improving crop quality whilst on the plant or following harvest;
e) reducing a biotic or an abiotic stress response in a plant, for example a response to heat and drought stress;
f) maintaining the freshness of plants or plant parts;
g) preventing seed germination; and
h) weed control.

7. The compound according to claim 1, wherein the compound is 3-[(2E)-3-(4-fluorophenyl)prop-2-enoyl]-4-hydroxy-6-methyl-2H-pyran-2-one, and wherein the compound is operative for modifying at least one physiological process of a plant or plant part selected from:
a) preventing or slowing food ripening or crop maturation;
b) preventing or slowing plant or plant part senescence;
c) preventing or slowing flower senescence;
d) improving crop quality whilst on the plant or following harvest;
e) reducing a biotic or an abiotic stress response in a plant, for example a response to heat and drought stress;
f) maintaining the freshness of plants or plant parts;
g) preventing seed germination; and
h) weed control.

8. A method of inhibiting a post-germination ethylene production response of a plant or plant part comprising contacting the plant or the plant part with a compound of formula (1), wherein formula (1) comprises: 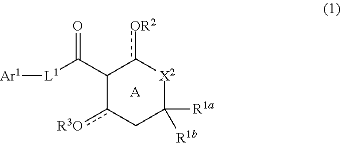
or a salt or tautomer thereof, wherein:
ring A is a six-membered aromatic or non-aromatic ring in which X¹ and X² are independently selected from O, CH, CH₂, CH(C₁₋₄ alkyl), and C(C₁₋₄ alkyl)₂;
R^{1a} and R^{1b} are independently selected from hydrogen and C₁₋₄ alkyl or R^{1a} and R^{1b} together form a carbonyl group with the carbon atom of ring A to which they are attached;
R² and R³ are independently selected from hydrogen and C₁₋₄ alkyl or are absent when the oxygen atom to which they are attached forms a carbonyl group with the carbon ring member of ring A;
L¹ is selected from a bond, —CH₂, —CH═CH— and —CH₂—CH₂—Ar¹ is a 5- or 6-membered carbocyclic or heterocyclic aromatic group optionally substituted by one or more substituents R⁴; and
R⁴ is selected from hydroxy, halogen, O—Ar², Hyd¹, O—Hyd¹, NH(Hyd¹) and N(Hyd¹)₂, wherein Hyd¹ is a C₁₋₄ hydrocarbon group optionally substituted with one or more fluorine atoms; and
Ar² is a 5- or 6-membered carbocyclic or heterocyclic aromatic group; and
whereby the compound is operative to inhibit a post-germination ethylene production response in the plant or in the plant part.

9. The method of claim 8, further comprising wherein the method is operative for modifying at least one physiological process of a plant or plant part selected from:
a) preventing or slowing the food ripening or crop maturation;
b) preventing or slowing plant or plant part senescence;
c) preventing or slowing flower senescence;
d) improving crop quality whilst on the plant or following harvest;
e) reducing a biotic or an abiotic stress response in a plant including a response to heat and drought stress;
f) maintaining the freshness of a plant or plant part; and
wherein the method comprises delivering a compound of formula (1) to the plant or plant part.

10. The method according to claim 8, wherein the compound of formula (1) is applied to a medium in which the plant or plant part is located, for subsequent uptake of the compound into the plant or plant part.

11. The method according to claim 8, wherein the compound of formula (1) is applied to a medium in which the plant or plant part is located, for subsequent uptake of the compound into the plant or plant part, and wherein the medium is a liquid.

12. The method according to claim 8, wherein the compound of formula (1) is applied to a medium in which the plant or plant part is located, for subsequent uptake of the compound into the plant or plant part, and wherein the medium is a solid.

13. The method according to claim 8, wherein the plant part is selected from the group consisting of: a leaf, stem, flower, seed, fruit or any combination thereof.

14. The method according to claim 8, wherein the compound is not 2,2,4-trimethyl-6-(3-phenylpropanoyl)cyclohexane-1,3,5-trione.

15. The method according to claim 8, wherein the compound is 5,5-dimethyl-2-(2-phenylacetyl) cyclohexane-1,3-dione.

16. The method according to claim 8, wherein the compound is 3-[(2E)-3-(4-fluorophenyl)prop-2-enoyl]-4-hydroxy-6-methyl-2H-pyran-2-one.

17. The method according to claim 8, wherein the compound of formula (1) is applied to a medium in which the plant or plant part is located, for subsequent uptake of the compound into the plant or plant part, and wherein the concentration of the compound applied to the medium in which the plant or plant part is located is between 0.005 μM and 50 μM.

18. The method according to claim 8, wherein the compound of formula (1) is applied to a medium in which the plant or plant part is located, for subsequent uptake of the compound into the plant or plant part, and wherein the concentration of the compound applied to the medium in which the plant or plant part is located is between 5 μM and 15 μM.

19. The method according to claim 8, further comprising whereby an execution of the method results in at least one of the following processes:
a) preventing or slowing food ripening or crop maturation;
b) preventing or slowing plant or plant part senescence;
c) preventing or slowing flower senescence;
d) improving crop quality whilst on the plant or following harvest;
e) reducing a biotic or an abiotic stress response in a plant, for example a response to heat and drought stress;
f) maintaining the freshness of plants or plant parts; and
wherein formula (1) is 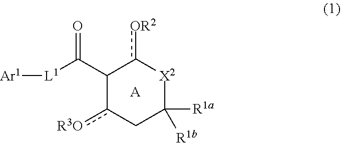
or a salt or tautomer thereof, wherein:
ring A is a six-membered aromatic or non-aromatic ring in which X¹ and X² are independently selected from O, CH, CH₂, CH(C₁₋₄ alkyl), and C(C₁₋₄ alkyl)₂;
R^{1a} and R^{1b} are independently selected from hydrogen and C₁₋₄ alkyl or R^{1a} and R^{1b} together form a carbonyl group with the carbon atom of ring A to which they are attached;
R² and R³ are independently selected from hydrogen and C₁₋₄ alkyl or are absent when the oxygen atom to which they are attached forms a carbonyl group with the carbon ring member of ring A;
L¹ is selected from a bond, —CH₂, —CH═CH— and —CH₂—CH₂—Ar¹ is a 5- or 6-membered carbocyclic or heterocyclic aromatic group optionally substituted by one or more substituents R⁴; and
R⁴ is selected from hydroxy, halogen, O—Ar², Hyd¹, O—Hyd¹, NH(Hyd¹) and N(Hyd¹)₂, wherein Hyd¹ is a C₁₋₄ hydrocarbon group optionally substituted with one or more fluorine atoms; and
Ar² is a 5- or 6-membered carbocyclic or heterocyclic aromatic group.
